(19) United States
(12) Patent Application Publication
(10) Pub. No.: US 2009/0177237 A1
(43) Pub. Date: Jul. 9, 2009

(54) CERVICAL SPINE IMPLANT SYSTEM AND METHOD

(75) Inventors: James F. Zucherman, San Francisco, CA (US); Ken Y. Hsu, San Francisco, CA (US)

Correspondence Address: FLIESLER MEYER LLP, 650 CALIFORNIA STREET, 14TH FLOOR, SAN FRANCISCO, CA, 94108, US

(73) Assignee: SPARTEK MEDICAL, INC., Alameda, CA (US)

(21) Appl. No.: 12/237,109

(22) Filed: Sep. 24, 2008

Related U.S. Application Data
(60) Provisional application No. 61/019,105, filed on Jan. 4, 2008.

(51) Int. Cl. A61B 17/70; A61B 17/04

(52) U.S. Cl. 606/280; 606/74; 606/286; 606/301

(57) ABSTRACT

Systems and methods for treating medical conditions affecting the spine using posterior plating techniques wherein two or more motion preservation plates are used to create joints between adjacent lateral masses. Additionally, embodiments of a facet spacer guide can be used to create a pilot hole in a lateral mass to facilitate screw fixation therein.

### CLAIM OF PRIORITY

This application claims priority to U.S. Provisional Application No. 61/019,105, filed Jan. 4, 2008, entitled “Cervical Spine Implant and Method” (Attorney Docket No. SPART-01034US0), which is incorporated herein by reference in its entirety.

### FIELD OF THE INVENTION

The present invention relates generally to spinal implants.

### BACKGROUND

The spine normally includes thirty-three stacked vertebrae which can be divided into five regions. The first seven vertebrae are called the cervical vertebrae and are located at the top of the vertebral column. They are identified, according to their position, as C1-C7. The next twelve vertebrae are called the thoracic vertebrae. These bones move with the ribs to form the rear anchor of the rib cage. They are identified, according to their position, as T1-T12. The next five vertebrae are called the lumbar vertebrae. These vertebrae help to support most of the body's weight. They are identified, according to their position, as L1-L5. The next region is called the sacrum and includes five vertebrae, S1-S5. Finally, the bottom of the vertebral column is called the coccyx. It consists of four vertebrae, Co1-Co4.

Each year, millions of people suffer from some type of instability of the spine. This spinal instability can be caused by, among other things, trauma, malignancy, congenital malformation or inflammatory diseases. Whatever the etiology, surgery is often necessary to remedy the spinal instability. In recent years, posterior plating (or rodding) utilizing lateral mass screw fixation has been accepted as an effective method for treating spinal instability.

Posterior plating utilizing lateral mass screw fixation generally involves coupling two or more vertebrae together using solid plates. These plates are fixated to the lateral masses of the vertebrae using screws. Several techniques of lateral mass screw insertion have been proposed in the past and are well known to those skilled in the relevant art. These lateral mass screw insertion techniques typically involve slight variations with respect to their starting point in the mass, degree of divergence from the midline, and sagittal plane orientation relative to the facet joint. Regardless of the specific screw insertion technique employed, the overall advantage of posterior plating using lateral mass fixation is that it provides equal or greater biomechanical stability when compared to other anterior plating techniques or traditional interspinous wiring techniques. It is particularly useful for patients whose spinous processes, laminae, and/or facets have been injured or are deficient.

### BRIEF DESCRIPTION OF THE DRAWINGS

The accompanying drawings, which are incorporated into and constitute a part of this specification, illustrate one or more embodiments and, together with the detailed description, serve to explain the principles and implementations of these embodiments of the invention. In the drawings:

FIG. 1A illustrates a side view of a healthy cervical spine.

FIG. 1B illustrates a side view of a cervical spine suffering from spinal stenosis.

FIG. 2 illustrates a rear view of a pair of cervical vertebrae.

FIG. 3A illustrates a side view of a facet spacer guide in accordance with an embodiment of the invention.

FIG. 3B illustrates a rear view of a facet spacer guide in accordance with an embodiment of the invention.

FIG. 4A illustrates a rear view of a facet spacer guide located between adjacent vertebrae in accordance with an embodiment of the invention.

FIG. 4B illustrates a side view of a facet spacer guide located between adjacent vertebrae in accordance with an embodiment of the invention.

FIG. 5 illustrates a side view of a facet spacer guide located between adjacent vertebrae in accordance with an embodiment of the invention.

FIG. 6A illustrates a perspective view of a facet spacer guide in accordance with an embodiment of the invention.

FIG. 6B illustrates a perspective view of a facet spacer guide in accordance with an embodiment of the invention.

FIG. 7 illustrates a rear view of conforming motion preservation plates for decompression and stabilization of the spine in accordance with an embodiment of the invention.

FIG. 8 illustrates a side view of conforming motion preservation plates for decompression and stabilization of the spine in accordance with an embodiment of the invention.

FIG. 9A illustrates a side view of conforming motion preservation plates for decompression and stabilization of the spine in accordance with an embodiment of the invention.

FIG. 9B illustrates a side view of conforming motion preservation plates for decompression and stabilization of the spine in accordance with an embodiment of the invention.

FIG. 9C illustrates a side view of conforming motion preservation plates for decompression and stabilization of the spine in accordance with an embodiment of the invention.

FIG. 10 illustrates a side view of conforming motion preservation plates for decompression and stabilization of the spine in accordance with an embodiment of the invention.

FIG. 11A illustrates a perspective view of a motion preservation plate for decompression and stabilization of the spine in accordance with an embodiment of the invention.

FIG. 11B illustrates a rear view of conforming motion preservation plates for decompression and stabilization of the spine in accordance with an embodiment of the invention.

FIG. 11C illustrates a side view of conforming motion preservation plates for decompression and stabilization of the spine in accordance with an embodiment of the invention.

FIG. 12A illustrates a rear view of motion preservation plates for decompression and stabilization of the spine in accordance with an embodiment of the invention.

FIG. 12B illustrates a side view of motion preservation plates for decompression and stabilization of the spine in accordance with an embodiment of the invention.

FIG. 12C illustrates a rear view of conforming motion preservation plates for decompression and stabilization of the spine in accordance with an embodiment of the invention.

FIG. 12D illustrates a side view of conforming motion preservation plates for decompression and stabilization of the spine in accordance with an embodiment of the invention.

FIG. 12E illustrates a rear view of a motion preservation plates for decompression and stabilization of the spine in accordance with an embodiment of the invention.

### DETAILED DESCRIPTION

Embodiments are described herein in the context of cervical spine implant systems and methods related thereto. Those of ordinary skill in the art will realize that the following detailed description is illustrative only and is not intended to be in any way limiting. Other embodiments of the present invention will readily suggest themselves to such skilled persons having the benefit of this disclosure. Reference will now be made in detail to implementations of embodiments of the present invention as illustrated in the accompanying drawings. The same reference indicators will be used throughout the drawings and the following detailed description to refer to the same or like parts.

Disclosed herein are systems and methods for treating medical conditions affecting the spine using posterior plating techniques. In the embodiments described below, systems and methods affecting the cervical region of the spine are disclosed. However, one of skill in the art may readily adapt the disclosed devices and methods in other regions of the spine without departing from the scope of the present invention.

As set forth in the foregoing background of the invention, posterior plating utilizing lateral mass screw fixation has become accepted as an effective method for treating spinal instability. While traditional posterior plating techniques utilizing lateral mass screw fixation have been found to be relatively effective in stabilizing a damaged spine, these techniques are less ideal for treating other medical conditions which negatively affect the cervical vertebrae. One such medical condition is spinal stenosis.

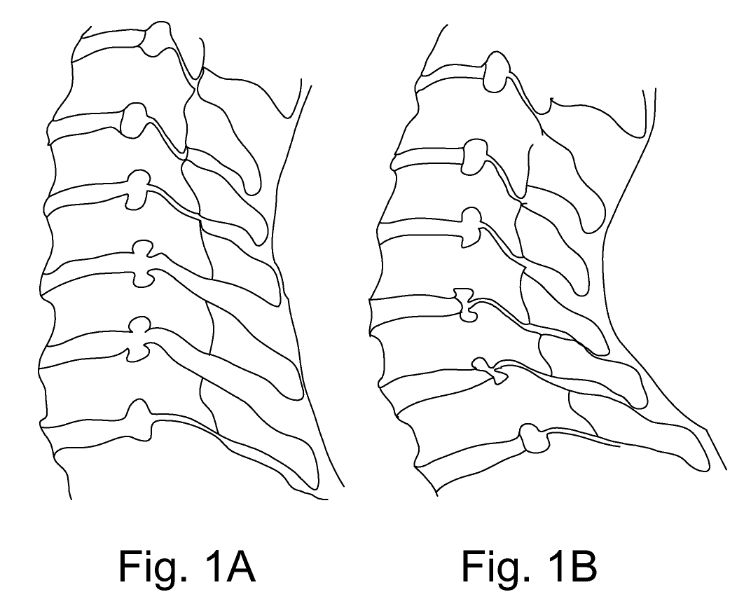

Spinal stenosis is a medical condition wherein one or more areas in the spine become narrowed. Referring to FIG. 1A, this figure illustrates a typical, healthy cervical column. FIG. 1B, on the other hand, illustrates the same cervical column suffering from spinal stenosis. The narrowing of the areas between the vertebrae can readily be seen when comparing the two cervical columns in these figures. This narrowing of the spine can place pressure on the spinal cord or nerves that branch out from the compressed areas, thereby causing pain and/or discomfort to the person suffering from spinal stenosis.

Traditional posterior plating techniques utilizing lateral mass screw fixation are typically not employed in treating patients suffering from spinal stenosis. One reason for this is the fact that while traditional posterior plating techniques increase the stability of the spine, such traditional techniques do so by locking the vertebrae into fixed positions relative to one another. Consequently, while vertebral stability may be achieved, mobility between the affected vertebrae may be lost forever. This may not be a desirable trade-off for patients suffering from spinal stenosis.

Given the loss of mobility between the affected vertebrae and the inherent difficulties present when physically executing the necessary surgical procedures, it was previously unexpected for a surgeon to utilize traditional posterior plating techniques to treat spinal conditions such as spinal stenosis. However, the inventions embodied herein include novel cervical spine implant systems and methods which will promote the use of posterior plating techniques when treating adverse spinal conditions such as spinal stenosis. Accordingly, the benefits of posterior plating techniques can be taken advantage of by a much greater range of patients suffering from a wide variety of ailments negatively affecting the spine.

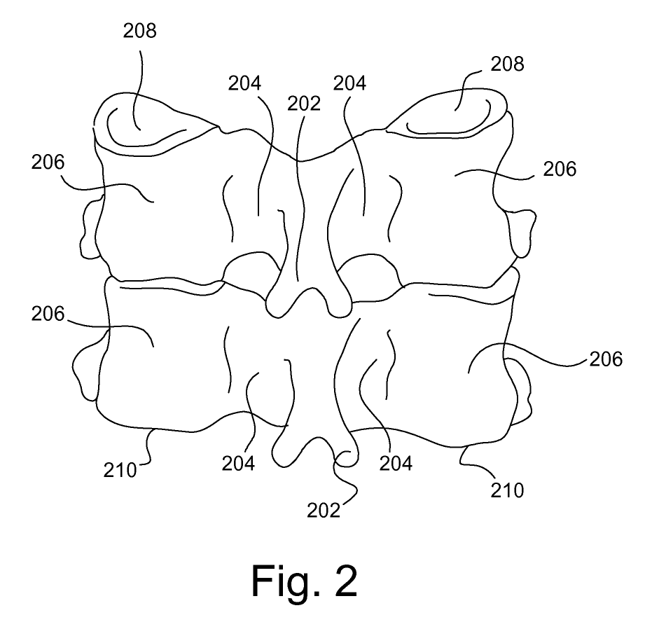

Turning now to FIG. 2, a representative pair of cervical vertebrae 200 is illustrated. As can be seen in FIG. 2, a typical cervical vertebrae 200 includes, among other things, a spinous process 202, lamina 204, lateral masses 206, and facets 208, 210 including the superior articular facets 208 and the inferior articular facets 210. The lateral mass 206 being the area lateral to the lamina 204 between the superior articular facet 208 and the inferior articular facets 210. During a typical posterior plating procedure, a patent is placed in the prone position and the patient's back is dissected to expose the underlying cervical vertebrae 200 using standard surgical techniques. Plates can then be secured to the adjacent lateral masses 206 of the cervical vertebrae 200 using any lateral mass screw insertion technique.

Different techniques for lateral mass screw insertion can include slight variations with respect to the starting point of such techniques in the lateral mass, degree of divergence from the midline, and sagittal plane orientation relative to the facet joint. Nevertheless, while various screw insertion techniques are well known by those skilled in the art, physically executing those techniques in the field can still prove to be difficult. This is especially true when attempting to insert a bone screw into a lateral mass 206 at a precise angle and location along the lateral mass 206. Accordingly, what is disclosed herein is a facet spacer guide that can be used to facilitate lateral mass screw fixation. It is noted that as the term “horizontal” refers to a horizontal orientation with respect to a human patient that is standing and “vertical” refers to a vertical orientation with respect to a patient that is standing with all embodiments of the invention set forth herein.

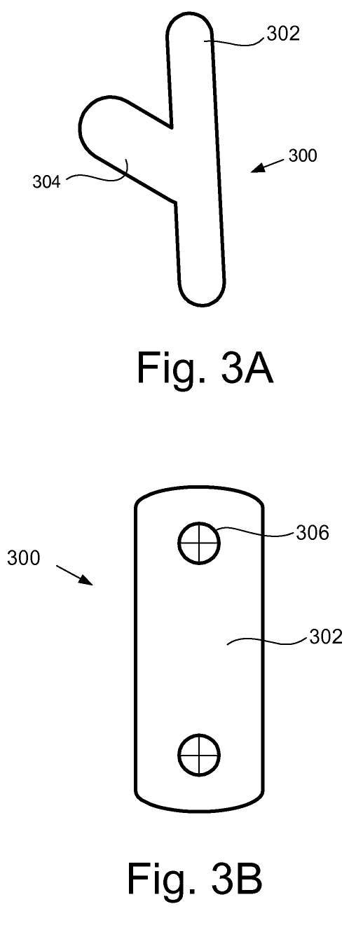

Referring now to FIGS. 3A and 3B, an embodiment of a facet spacer guide, which can be used to facilitate lateral mass screw fixation, is illustrated. The facet spacer guide shown in FIGS. 3A and 3B facilitates surgical procedures by taking advantage of the nature of the cervical facet anatomy. Referring to FIG. 3A, in one embodiment of the invention, the facet spacer guide 300 is generally “y-shaped” and includes a base plate 302 having an intervertebral wedge 304. Prior to insertion of the bone screw into a lateral mass 206, the facet spacer guide 300 can be used as a template to create a pilot hole within the lateral mass 206 at the desired angle and location.

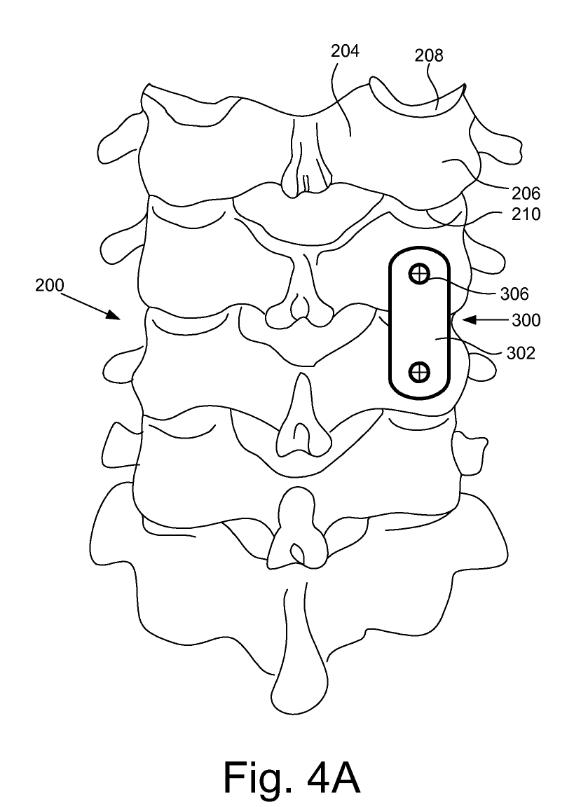

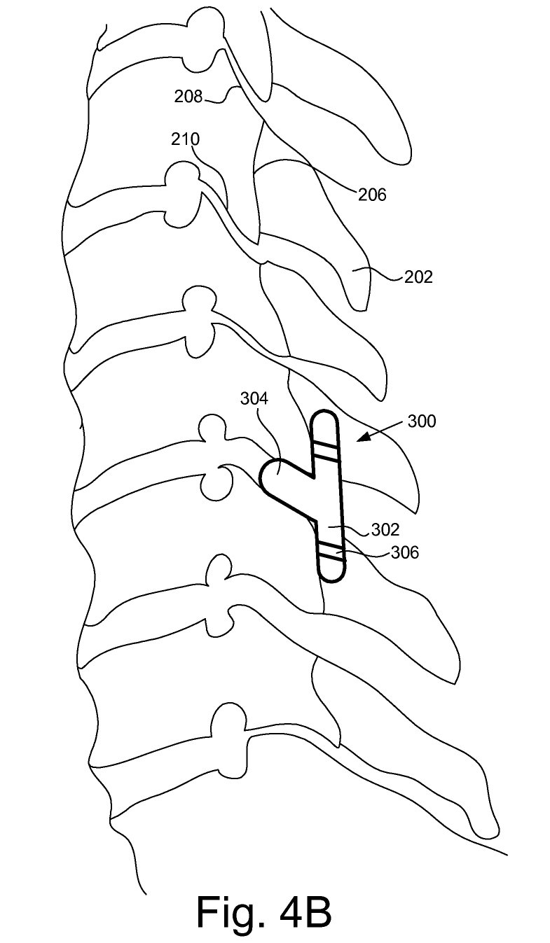

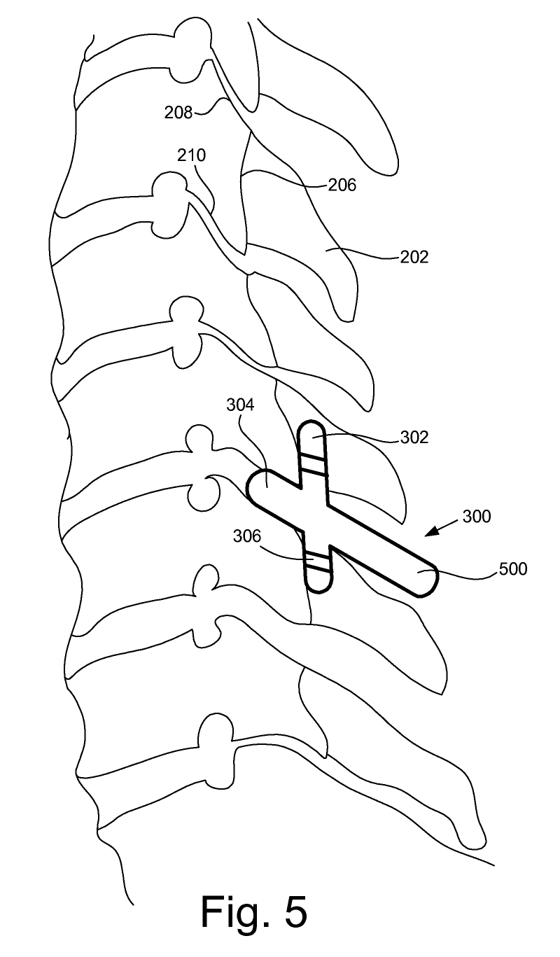

Accordingly, in use, the intervertebral wedge 304 can be inserted in between the superior articular facet 208 of a vertebra 200 and the inferior articular facet 210 of an adjacent vertebra 200 as shown in FIG. 4B. The base plate 302 is preferably aligned with and placed adjacent to the posterior surfaces of the inferior 210 and superior lateral masses in its deployment position as shown in FIGS. 4A and 4B. FIG. 4A shows a rear view of the facet spacer guide 300 inserted between a pair of vertebrae 200. FIG. 4B shows a side view of the same facet spacer guide 300 inserted between a pair of vertebrae 200. The angle of the second plate 304 in relation to the base plate 302 can be any angle which facilitates the process of positioning the facet spacer guide 300 in between adjacent vertebrae as shown in FIGS. 4A and 4B according to the particular patient's anatomy. In this embodiment, the intervertebral wedge 304 is attached to the base plate 302 at a central location. The intevertebral wedge 304, however, can also be attached to the base plate 302 at any other position along the base plate 302. In another embodiment, the width of the intervertebral wedge 304 can be varied to control the desired level of distraction between the vertebrae 200. In yet another embodiment, the base plate 302 includes a plurality of interchangeable intervertebral wedges 304, wherein each intervertebral wedge can have different sizes and/or shapes and provide different amounts of distraction and wherein each wedge can be positioned between the vertebrae. Still additionally, in another embodiment, a plurality of guides 300 is provided, with each guide 300 having a wedge 304 having a different size and/or shape and thus each wedge 304 can provide a different amount of distraction between the vertebrae.

Referring again to FIG. 4A, the base plate 302 also includes at least one aperture 306 wherein a drill, awl or any other device for creating a hole in the lateral mass 206 can be inserted. Once the facet spacer guide 300 is properly positioned at the desired location within the cervical column, the hole creating device can be inserted through the aperture 306 and into the lateral mass 206, thereby creating a pilot hole within the lateral mass 206 prior to screw fixation. Once the pilot hole is created, the facet spacer guide 300 can be removed and the lateral mass screw fixation procedure can begin.

In an embodiment, the apertures 306 can be placed at any location along the base plate 302 corresponding to the desired location for the screw insertion point along the lateral mass 206. In another embodiment, a plurality of apertures 306 can be included in the facet spacer guide 300, each aperture 306 corresponding to different desired insertion positions associated with different proposed techniques for lateral mass screw insertion. Another benefit of using the facet spacer guide 300 is that it allows the surgeon to introduce two or more screws into adjacent vertebrae at the same angles when performing lateral mass screw fixation procedures. Accordingly, in another embodiment, the apertures 306 can also be angled within the base plate 302 to correspond to a desired angle of insertion associated with a desired technique for lateral mass screw insertion.

In another embodiment, the facet spacer guide 300 can also include a handle 500 as shown in FIG. 5. The handle 500 can be used when inserting the facet spacer guide 300 between adjacent facets 208, 210 to facilitate the insertion process. The handle 500 can also be held when creating the pilot holes to stabilize the facet spacer guide 300 during use.

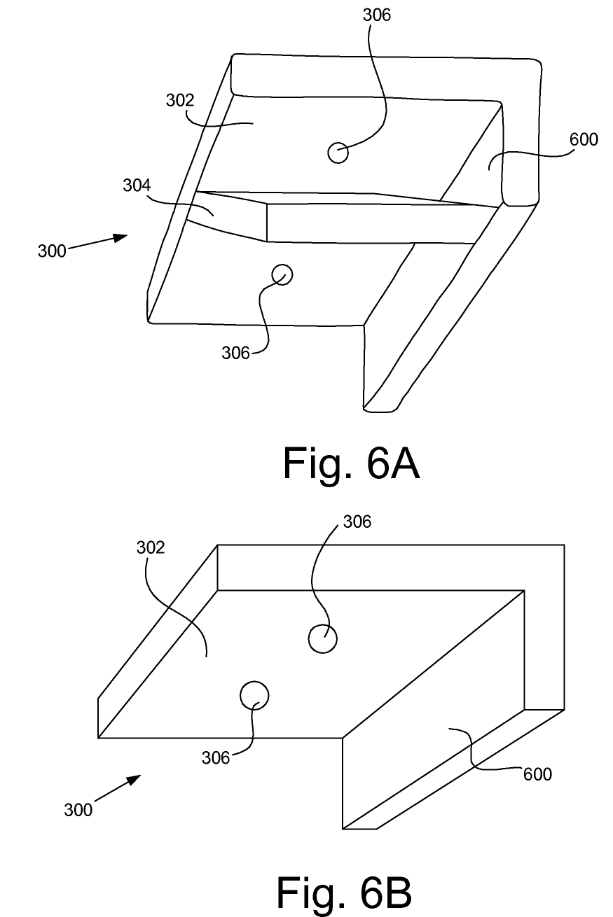

Referring now to FIG. 6A, in this embodiment, the facet spacer guide 300, includes the base plate 302, an intervertebral wedge 304, apertures 306 and a side plate 600. The side plate 600 can be used to prevent the spacer guide 300 from slipping medially during use. In another embodiment, the facet spacer guide 300 can be used with a side plate 600 but without the intervertebral wedge 304 as illustrated in FIG. 6B. While some embodiments and applications of the facet spacer guide have been shown and described above, it would be apparent to those skilled in the art having the benefit of this disclosure that many more modifications than mentioned above are possible without departing from the inventive concepts herein.

Once the pilot holes are created in accordance with the embodiments of the invention set forth above and the vertebrae are positioned relative to each other at the desired locations using standard operating procedures, plates can be fixed to the lateral masses utilizing an appropriate lateral mass screw fixation technique. Traditional posterior plating techniques utilize solid plates to stabilize the spine. However, while the use of solid plates may increase the stability of the affected vertebrae, such solid plates also serve to lock the affected vertebrae to a single position relative to each other. Consequently, mobility of the affected vertebrae may be lost forever. An object of the present invention is to apply posterior plating techniques to stabilize the spine while preserving mobility between the affected vertebrae.

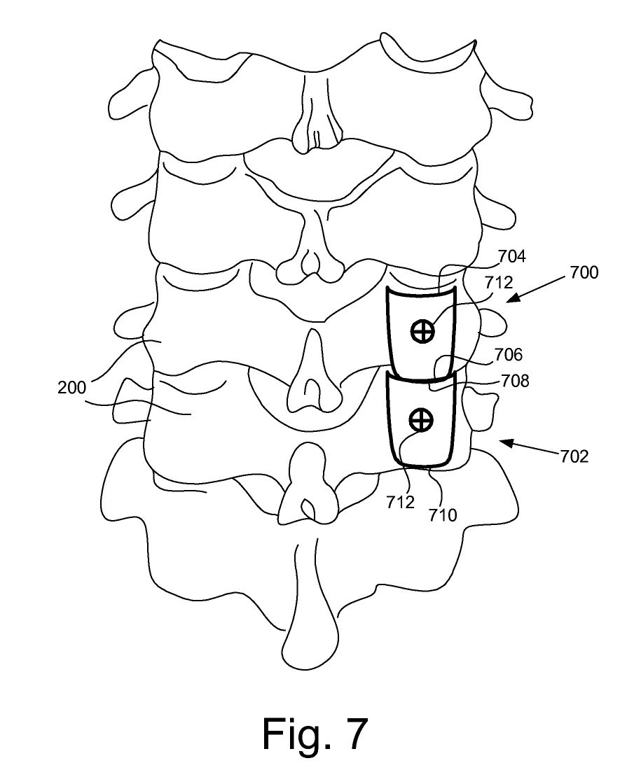

Referring now to FIG. 7, motion preservation plates 700, 702 for decompression and stabilization of the spine in accordance with an embodiment of the present invention are illustrated. This embodiment of the invention can be seen as including a first motion preservation plate 700 and a second motion preservation plate 702, each plate attached to adjacent vertebrae 200. Each motion preservation plate 700, 702 includes a concave, arcuate top surface 704, 708, a convex, arcuate bottom surface 706, 710 and an aperture 708 for accepting a screw. In its deployed configuration, the motion preservation plates 700, 702 are fixed to adjacent vertebrae 200 using an appropriate lateral mass screw insertion technique, the bottom surface 706 of the first motion preservation plate 700 being engaged to the top surface 708 of the second motion preservation plate 702. In this configuration, the motion preservation plates 700, 702 can cause distraction, and prevent compression of the adjacent vertebrae 200 while preserving the ability of the affected vertebrae 200 to have forward, backward and lateral movements. In other words, the motion preservation plates 700, 702 can create an artificial joint between two adjacent vertebrae 200 to prevent the vertebrae 200 from becoming compressed (as shown in FIG. 1B). Accordingly, the motion preservation plates 700, 702 can remain in sliding engagement as the affected vertebrae 200 are moved from side-to-side, and also front to back rocking engagement, and also can even become temporarily separated as the first motion preservation plate 700 is moved forward relative to the second motion preservation plate 702 in use during patient flexion.

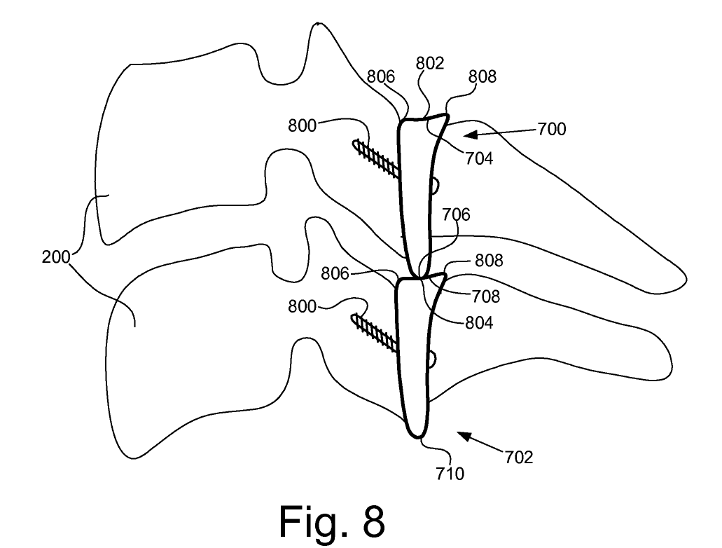

Referring now to FIG. 8, a further embodiment of the invention is shown. FIG. 8 illustrates a first motion preservation plate 700 and a second motion preservation plate 702, both of which are attached to adjacent vertebrae 200 using screws 800. In this embodiment, the top surface 704, 708 of each motion preservation plate 700, 702 includes a U-shaped recess (or “valley”) 802, 804. The motion preservation plates 700, 702 are also tapered, the top surfaces 704, 708 being wider than the bottom surfaces 706, 710 thereby allowing the bottom surface 706 of the first motion preservation plate 700 to be engaged to the top surface 708 of the second motion configuration plate 702 within the U-shaped recess 804. In this configuration, the first motion preservation plate 700 can be confined between the back 806 and front 808 edges of the U-shaped recess 804 on the top surface 708 of the second motion preservation plate 702, thereby preserving the alignment of the first motion preservation plate 700 with respect to the second motion preservation plate 702 in use within a patient. As with the embodiment of the motion preservation plates 700, 702 illustrated in FIG. 7, the top surfaces 704, 708 of the motion preservation plates 700, 702 illustrated in FIG. 8 can be concave while the bottom surfaces 706, 710 are convex, thereby allowing the motion preservation plates 700, 702 to remain in sliding and/or rocking engagement as the adjacent vertebrae 200 are moved from side-to-side. In an embodiment, the motion preservation plates 700, 702 can become temporarily detached as the first motion preservation plate 700 is moved forward relative to the second motion preservation plate 702 in use during patient flexion. In another embodiment, the vertical depth of the U-shaped recess 804 of the second motion preservation plate 702 can be increased and/or otherwise configured to prevent the motion preservation plates 700, 702 from becoming separated as the first motion preservation plate 700 is moved forward relative to the second motion preservation plate 702 during use within a patient. In still another embodiment of the invention the bottom of upper plate can be concave and the top of the lower plate can be convex, the inverse of the embodiment of FIG. 8, and be within the spirit and scope of the invention. Additionally, other mating arrangements between the two plates can be within the spirit and scope of the invention.

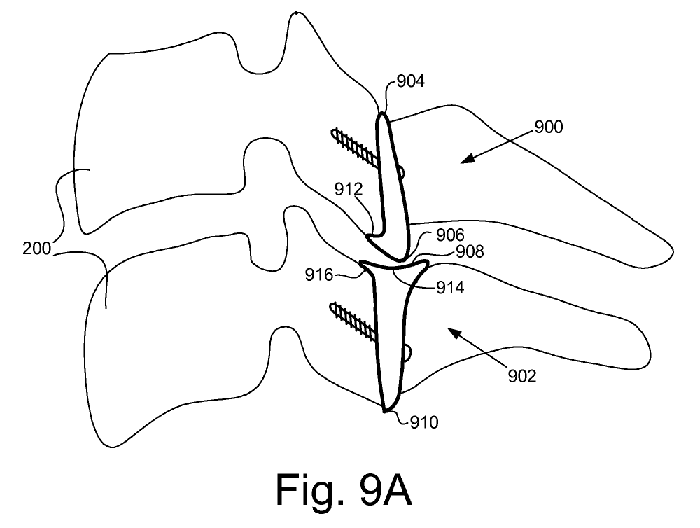

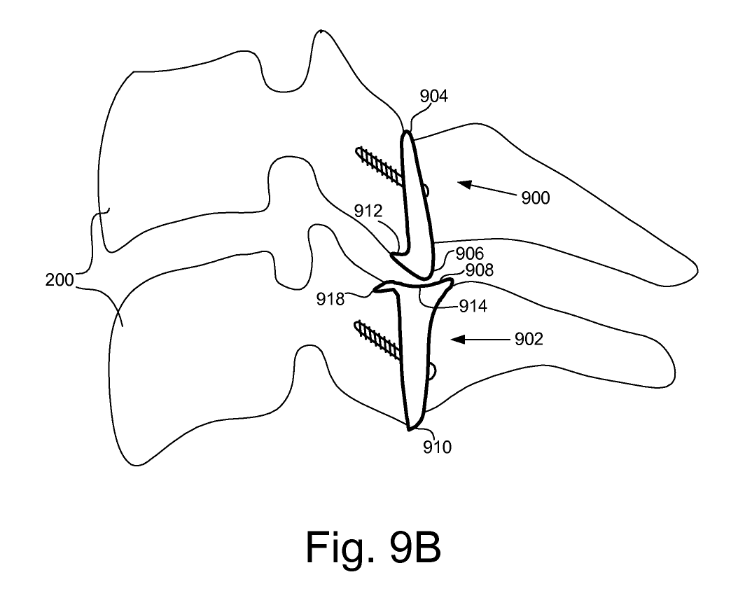

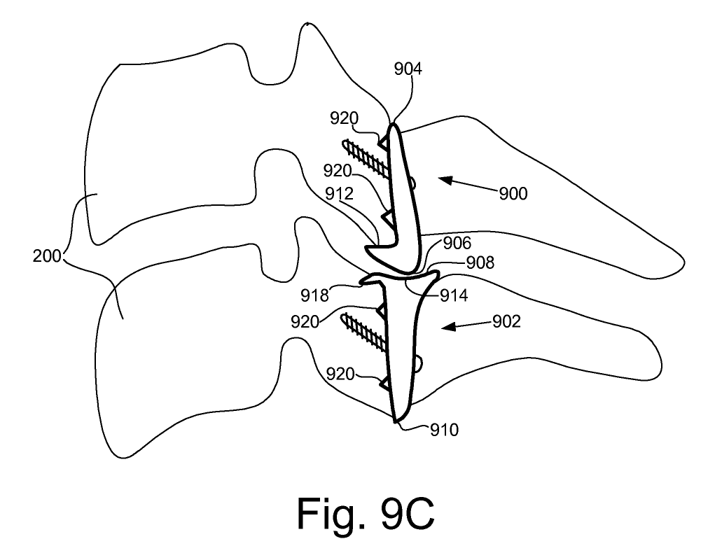

FIG. 9A shows another embodiment of the invention. As shown in FIG. 9A, a first motion preservation plate 900, having a first end 904 and a second end 906, and a second motion preservation plate 902, also having a first end 908 and a second end 910, are attached to adjacent vertebrae 200 to decompress and stabilize the spine. In this embodiment, the first motion preservation plate 900 is also tapered, wherein the bottom portion of the first motion preservation plate 900 is wider than the top portion of the first motion preservation plate 900. The first motion preservation plate 900 also includes a buttress 912 on the back surface (which can also be referred to as the “anterior engagement surface”) of the first motion preservation plate 900 which can be placed proximal to the posterior aspect of the vertebra 200 to increase purchase on the bone. In this embodiment, the buttress is located along the bottom surface 906 of the first motion preservation plate 900 and placed adjacent to the inferior articular facet 210. In an embodiment, the bottom surface 906 of the first motion preservation plate 900 can be convex and arcuate (as shown in FIG. 7 for plate 700), thereby creating a rounded bottom surface 906 which can be received by and placed in sliding and/or rocking engagement with the top surface 908 of the second motion preservation plate 902.

Referring again to FIG. 9A, the second motion preservation plate 902 can be seen as including a top surface 908 having a U-shaped recess (or “valley” or “depression”) 914. The top surface 908 of the second motion preservation plate 902 can also be concave and arcuate as shown in FIG. 7 for plate 702. The top surface 908 of the second motion preservation plate 902 is thus configured to receive and be placed in sliding and/or rocking engagement with the bottom surface 906 of the first motion preservation plate 900. The second motion preservation plate 902 can also be tapered, wherein the top portion of the second motion preservation plate 902 is wider than the bottom portion of the second motion preservation plate 902. In an embodiment, the second motion preservation plate 902 includes a buttress 916 on the back surface of the first motion preservation plate 900 which can be placed proximal to the superior articular facet 208 to increase purchase on the bone. In yet another embodiment, the second motion preservation plate 902 includes a spike 918 as opposed to a buttress 916 (as shown in FIG. 9B). The spike 918 of this embodiment can be inserted directly into the bone to further increase the purchase on the superior articular facet 408. In yet another embodiment, the motion preservation plates 900, 902 can also include additional spikes 920 on their respective anterior engagement surfaces to further increase purchase on the bone as shown in FIG. 9C.

Similar to the previously described embodiments of the motion preservation plates 700, 702 illustrated in FIG. 7, the top surface 908 of the second motion preservation plate 902 illustrated in FIGS. 9A-9C can be concave while the bottom surface 906 of the first motion preservation plate 900 is convex, thereby allowing the motion preservation plates 900, 902 to remain in sliding and/or rocking engagement as the adjacent vertebrae 200 are moved from side-to-side and/or forward and backward. In an embodiment, the motion preservation plates 900, 902 can become temporarily separated as the first motion preservation plate 900 is moved forward relative to the second motion preservation plate 902 in use. In another embodiment, the vertical depth of the U-shaped recess 908 of the second motion preservation plate 902 can be increased and/or otherwise configured to prevent the motion preservation plates 900, 902 from becoming separated as the first motion preservation plate 900 is moved forward relative to the second motion preservation plate 902 during use within a patient. Still further, in another embodiment, the bottom engaging surface of the upper plate 900 can be concave and mated with a concave top engaging surface of the lower plate 902.

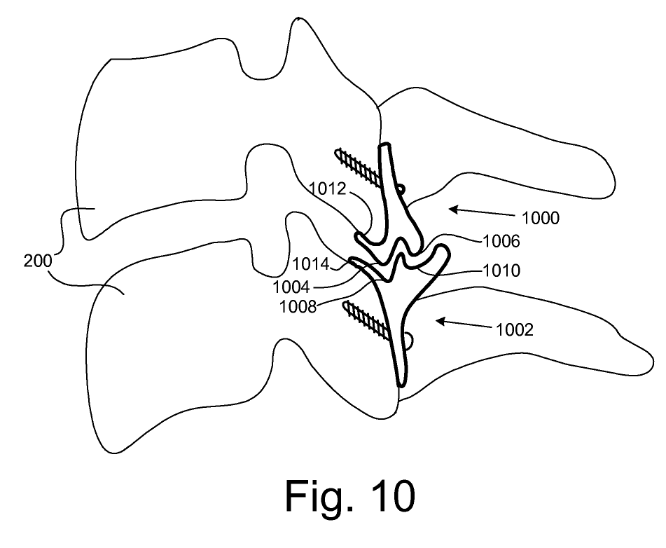

FIG. 10 illustrates yet another embodiment of the invention. In this embodiment, similar to motion preservation plates 900, 902 illustrated in FIG. 9A, a first motion preservation plate 1000 and a second motion preservation plate 1002 are attached to adjacent vertebrae 200 to decompress and stabilize the spine. In this embodiment, the first motion preservation plate 1000 includes two rounded bottom surfaces 1004, 1006 that can be mated to two corresponding recesses (or “valleys” or depressions”) 1008, 1010 located on the second motion preservation plate 1002. Both motion preservation plates 1000, 1002 also include buttresses 1012, 1014 with tapered ends on the anterior engagement surfaces of the respective plates to increase purchase on the bone. This configuration for the motion preservation plates 1000, 1002 further facilitates sliding and/or rocking engagement between the motion preservation plates 1000, 1002 in use as the adjacent vertebrae 200 are moved from side-to-side and/or forward and backward.

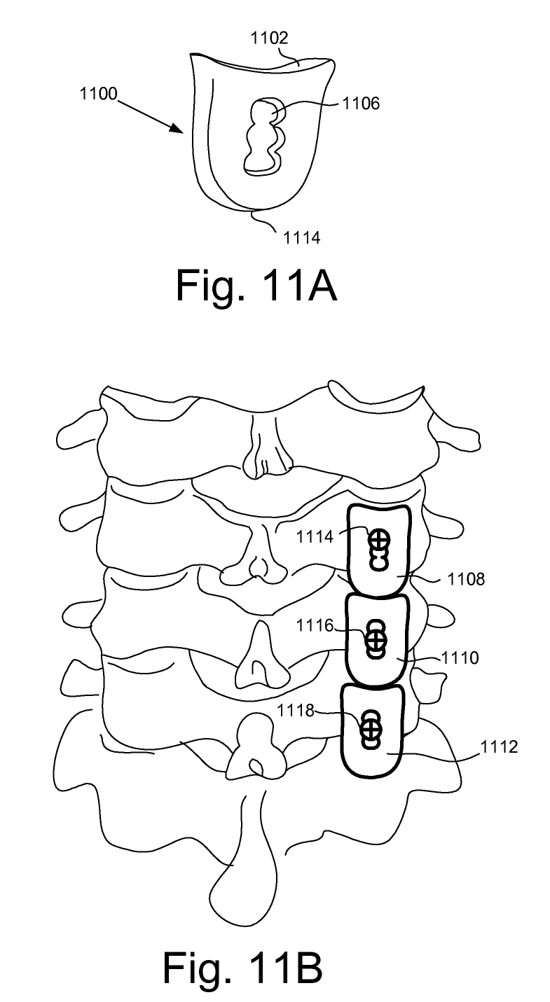

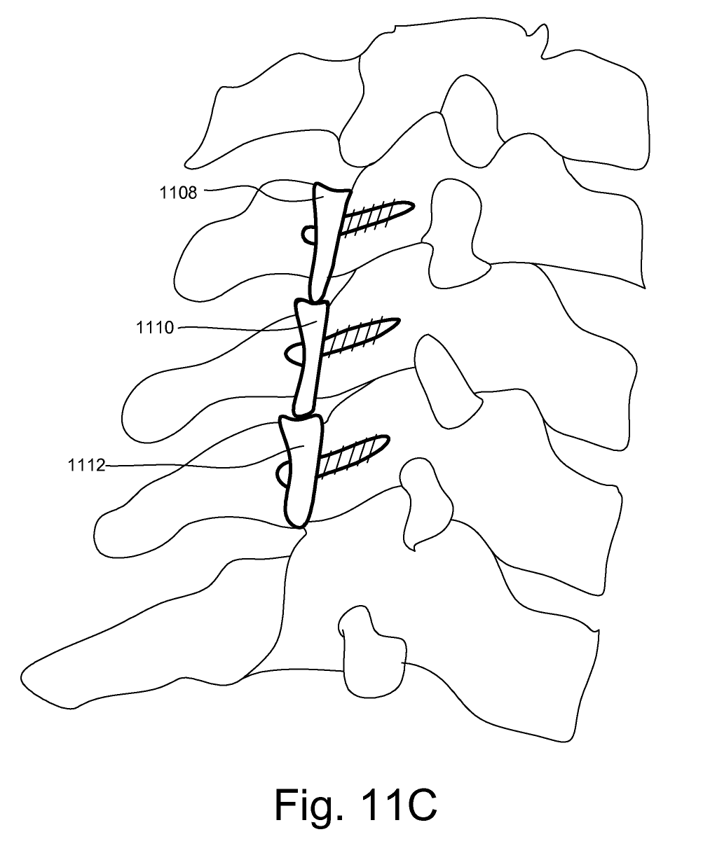

FIGS. 11A-11C illustrate another embodiment of the invention. Referring now to FIG. 11A, this embodiment of a motion preservation plate 1100 is similar to the motion preservation plates 700, 702 illustrated in FIG. 7. Both types of motion preservation plates 700, 1110 can be seen as including a concave, arcuate top surface 1102 and a convex, arcuate bottom surface 1104 and both types of motion preservation plates 700, 1110 function in a similar manner. Alternatively, the top surface of the plate can be convex and the lower surface of the plate can be concave. Further, in all embodiments of the plate of the invention, concave surfaces can be concave in a front to back and/or side to side orientation with respect to the plate, and convex surfaces can be convex in a front to back and/or side to side orientation of the plate. In this embodiment, however, instead of including a single aperture for accepting a screw, the motion preservation plate 1100 includes nested screw slots 1106 for multilevel use. For example, as shown in FIG. 11B, a screw 1114 can be inserted into the uppermost slot of a nested screw slot 1106 of a first motion preservation plate, while other screws 1116, 1118 can be inserted into the central slot of a second motion preservation plate 1110 and a central slot of a third motion preservation plates 1112. Consequently, the desired level of distraction between each vertebrae can be varied without changing the insertion point for the screw or the overall size of each motion preservation plate 1100. In an embodiment, as shown in FIGS. 11B-11C, a plurality of motion preservation plates 1108, 1110 and 1112 can be arranged in a series along a cervical column. It is noted that the embodiments of the invention shown in FIGS. 7-10 and described above can also include a plurality of motion preservation plates arranged in a series along a cervical column.

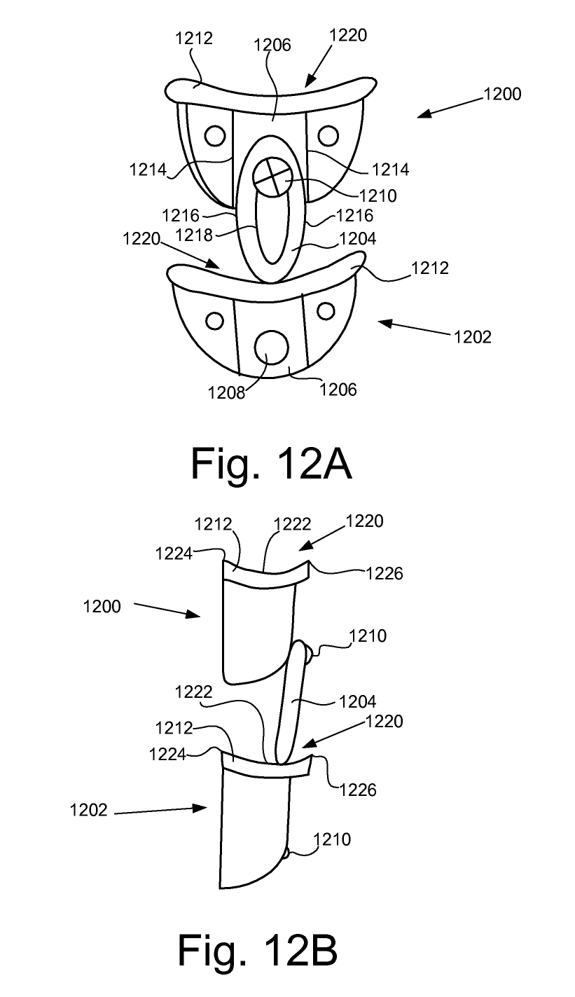

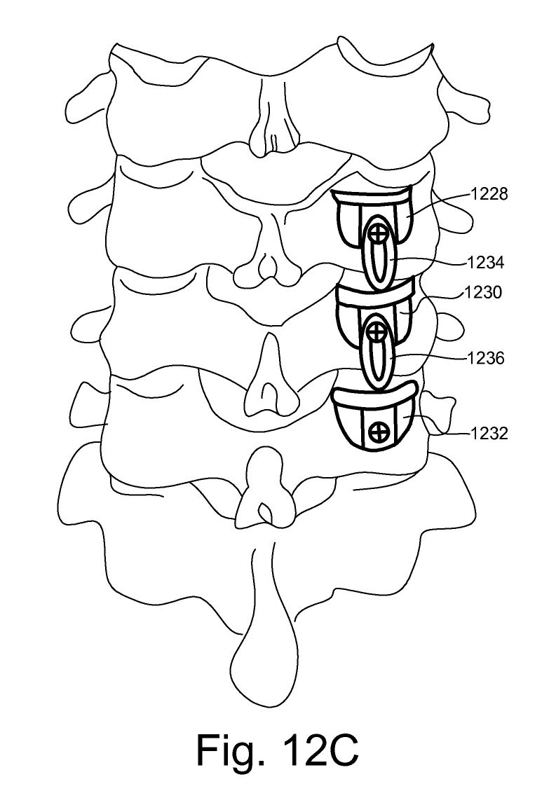

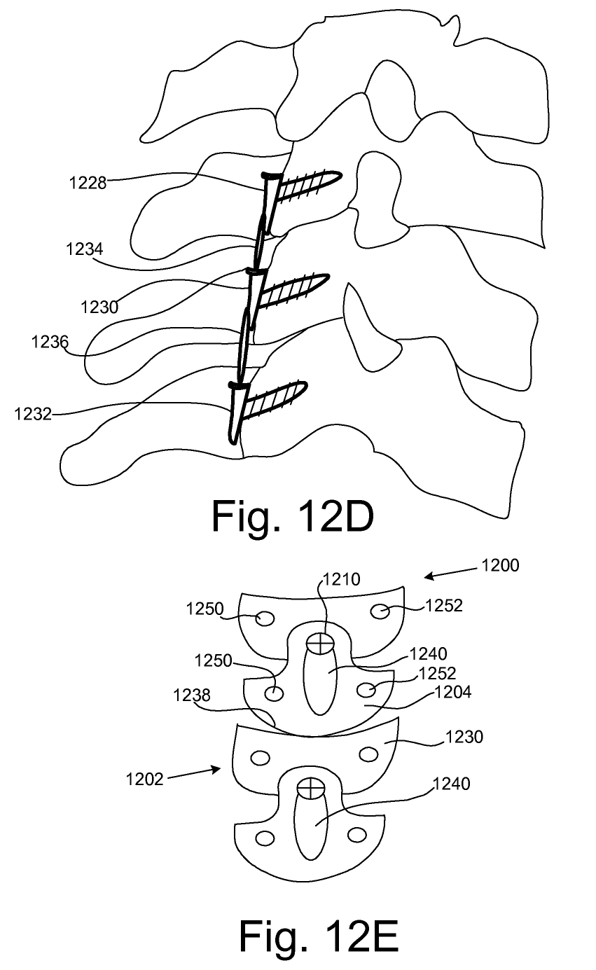

FIG. 12A illustrates another embodiment of the invention. This embodiment includes a first motion preservation plate 1200, a second motion preservation plate 1202 and a sliding strut 1204 to decompress and stabilize the spine. In this embodiment, the motion preservation plates 1200, 1202 each include a channel 1206 for accepting the sliding strut 1204, an aperture 1208 within the channel 1206 for accepting a screw 1210 and a horizontal ledge 1212 for accepting a sliding strut 1204. The sliding strut 1204 of this embodiment has the shape of an oval ring and can be inserted into the channel 1206 of the first motion preservation 1200 wherein the channel 1206 walls 1214 engage the sides 1216 of the sliding strut 1204 to help prevent horizontal movement of the sliding strut 1204 during use within a patient. The sliding strut 1204 can be attached to the first motion preservation plate 1202 through the use of a screw 1210, the same screw 1210 also being used to secure the first motion preservation plate 1202 to a vertebra 200. The sliding strut 1204 can be attached to the first motion preservation plate 1202 anywhere along its inner ring 1218, thereby allowing the user to vary the level of distraction between the first motion preservation plate 1200 and the second motion preservation plate 1202.

In the deployed configuration of this embodiment, the sliding strut 1204 is adapted to be received by and engaged to the top surface 1220 of the second motion preservation plate 1202 in sliding and/or rocking engagement. In this configuration, the top surface 1220 of the second motion preservation plate 1202 can be concave and arcuate to allow for sliding lateral movement and/or rocking motion between the sliding strut 1204 and the top surface 1220 of the second motion preservation plate 1202. Accordingly, as with the motion preservation plates 700, 702 illustrated in FIG. 7, the sliding strut 1204 attached to the first motion preservation plate 1200 can remain in sliding and/or rocking engagement with the second motion preservation plate 1202 as the adjacent vertebrae 200 moved from side-to-side and/or forward to backward in use within a patient. It is noted that apertures 1250, 1252 in the plates can also receive screws to secure the plate to the bone of the patient.

Referring now to FIG. 12B, a side view of the motion preservation plates 1200, 1202 is illustrated. As can be seen in FIG. 12B, the horizontal ledge 1212 can include a top surface 1220 having a U-shaped recess 1222. The top surface 1220 of the second motion preservation plate 1202 can engage the sliding strut 1204 attached to the first motion preservation plate 1200 within the U-shaped recess. In this configuration, the sliding strut 1204 can be confined between the back 1224 and front 1226 edges of the U-shaped recess 1222 on the top surface 1220 of the second motion preservation plate 1200. As shown in FIGS. 12C and 12D, a plurality of motion preservation plates 1228, 1230, 1232 and sliding struts 1234, 1236 can be also be arranged in a series along a cervical column.

As set forth above, the sliding strut 1204 of the embodiment of the invention illustrated in FIG. 12A has the shape of an oval ring. Nevertheless, it is envisioned that the sliding strut 1204 may have other shapes and configurations. For example, as shown in FIG. 12E, an embodiment of the sliding strut 1204 is illustrated as having the shape of an anchor, the sliding strut 1204 including a wide, rounded bottom surface 1238 which can be engaged to the second motion preservation plate 1202 and an internal oval ring 1240 through which a screw 1210 can be used to attach the sliding strut 1204 to the first motion preservation plate 1202.

It is noted that in the embodiments of the motion preservation plates illustrated in FIGS. 7-12E the anterior engagement surfaces (the plate surfaces placed adjacent to the posterior aspects of the vertebrae) of the motion preservation plates can be configured to lie flush with the surface of the lateral masses of the vertebrae. It is further noted that the size of the motion preservation plates can also be adjusted based on the desired level of distraction between the adjacent vertebrae 200. Accordingly, larger motion preservation plates having a greater vertical height can be used when it is desired to increase the amount of distraction between adjacent vertebrae.

The different embodiments of the facet spacer guides and motion preservation plates described herein can all be made from medical grade metals such as titanium, stainless steel, cobalt chrome, and alloys thereof, or other suitable materials such as ceramics, PEEK, polymers, copolymers, blends, and composites of polymers having similar high strength and biocompatible properties. The engagement surfaces for both devices may also be treated to facilitate fixation to the posterior surfaces of the cervical vertebral bodies. The surfaces may, for example, be provided with a porous titanium surface, plasma-sprayed titanium or similar surface that promotes bone growth and enhances fixation to the vertebral body. The anterior engagement surfaces of the devices may also be provided with surface features, such as roughening or additional spikes to enhance fixation. The other surfaces are preferably smooth and radiussed to reduce trauma to surrounding tissues.

The foregoing description of embodiments of the present invention has been provided for the purposes of illustration and description. It is not intended to be exhaustive or to limit the invention to the precise forms disclosed. Many embodiments were chosen and described in order to best explain the principles of the invention and its practical application, thereby enabling others skilled in the art to understand the invention for various embodiments and with various modifications that are suited to the particular use contemplated. In particular, the described devices may be used in all regions of the spine including the cervical, thoracic and lumbar regions. It is intended that the scope of the invention be defined by the claims and their equivalents.

1. A posterior plating device to position vertebrae, said device comprising:
a first motion preservation plate and a second motion preservation plate, each motion preservation plate being adapted to be attached to lateral masses of adjacent vertebrae;
wherein each motion preservation plate includes a top surface and a bottom surface;
wherein the bottom surface of the first motion preservation plate is in sliding engagement with the top surface of the second motion preservation plate to distract adjacent vertebrae; and
wherein at least one motion preservation plate includes a buttress on the anterior engagement surface of the at least one motion preservation plate to increase purchase on a bone.

2. The device of claim 1 wherein at least on buttress is a spike that is inserted directly into the bone.

3. The posterior plating device of claim 1, wherein at least one of the motion preservation plates includes nested screw slots.

4. The posterior plating device of claim 1, wherein the bottom surface of each motion preservation plate is convex.

5. The posterior plating device of claim 1, wherein the top surface of each motion preservation plate is concave to accept the bottom surface of an adjacent motion preservation plate.

6. The posterior plating device of claim 1, wherein each motion preservation plate is tapered, the top surface being wider than the bottom surface.

7. The posterior plating device of claim 1 comprising at least three motion preservation plates arranged in a series to distract adjacent vertebrae.

8. The device of claim 1 comprising at least one spike that is inserted directly in the bone on the anterior engagement surface of each motion preservation plate.

9. The device of claim 1 wherein the first motion preservation plate comprises two rounded bottom surfaces that are mated to two recesses located on the top surface of the second motion preservation plate.

10. A posterior plating device to position vertebrae, said device comprising:
a first motion preservation plate and a second motion preservation plate, each motion preservation plate being adapted to be attached to adjacent lateral masses of the vertebrae;
wherein each motion preservation plate includes a top surface and a bottom surface;
a sliding strut attached to the posterior aspect of the first motion preservation plate, said sliding strut having a top surface and a bottom surface; and
wherein the bottom surface of the sliding strut is in sliding engagement with the top surface of the second motion preservation plate to distract the adjacent vertebrae.

11. The posterior plating device of claim 7, wherein at least one motion preservation plate includes a channel to accept the sliding strut.

12. The posterior plating device of claim 7, wherein the top surface of each motion preservation plate is concave.

13. The posterior plating device of claim 7, wherein each motion preservation plate is tapered, the top surface being wider than the bottom surface.

14. The posterior plating device of claim 7, the sliding strut having the shape of an oval ring.

15. The posterior plating device of claim 7, the sliding strut having the shape of an anchor, wherein the bottom surface of the sliding strut is wider than the top surface of the sliding strut.

16. The posterior plating device of claim 7, wherein at least one motion preservation plate includes nested screw slots.

17. The posterior plating device of claim 7, further comprising at least three motion preservation plates arranged in a series on adjacent vertebrae, each motion preservation plate being distracted from the adjacent motion preservation plate through the use of sliding struts.

18. A facet spacer guide to facilitate lateral mass screw insertion techniques, said facet spacer guide comprising:
a base plate having at least one intervertebral wedge and at least one aperture;
said facet spacer guide adapted to allow the base plate to be located adjacent to posterior aspects of lateral masses of adjacent vertebrae while the at least one intervertebral wedge is placed between a superior articular facet and a inferior articular facet of the adjacent vertebrae; and
wherein the at least one aperture can accept a device to create a hole.

19. The facet spacer guide of claim 1 further comprising a handle.

20. The facet spacer guide of claim 1 further comprising a side plate.

21. The facet spacer guide of claim 1 further comprising a plurality of apertures.

22. The facet spacer guide of claim 1 further comprising a plurality of intervertebral wedges.

23. A posterior plating device, said device comprising:
a first motion preservation plate and a second motion preservation plate, each motion preservation plate being adapted to be attached to lateral masses of adjacent vertebrae;
wherein each motion preservation plate includes a top surface and a bottom surface; and
wherein the bottom surface of the first motion preservation plate is in engagement with the top surface of the second motion preservation plate to position adjacent vertebrae.
